(19) United States
(12) Patent Application Publication
(10) Pub. No.: US 2014/0023604 A1
(43) Pub. Date: Jan. 23, 2014

(54) EXTERNAL SKIN PREPARATION AND PRODUCTION METHOD OF SAME

(71) Applicant: Naoko Ito, Tokyo (JP)

(72) Inventor: Naoko Ito, Tokyo (JP)

(73) Assignee: SHOWA DENKO K.K., Minato-ku, Tokyo (JP)

(21) Appl. No.: 14/009,820

(22) PCT Filed: May 16, 2012
(86) PCT No.: PCT/JP2012/063106

§ 371 Date: Oct. 4, 2013

(30) Foreign Application Priority Data
Jun. 9, 2011 (JP) 2011-129279

(51) Int. Cl. A61K 8/55; A61Q 19/02

(52) U.S. Cl. CPC A61K 8/55; A61Q 19/02
USPC 424/62

(57) ABSTRACT

Provided are: an external skin preparation that contains a whitening agent in the form of a resorcinol derivative and causes minimal irritation of the skin, and a production method thereof. The external skin preparation contains 0.1% by weight to 5% by weight of the resorcinol derivative and 0.01% by weight to 5% by weight of the ascorbyl 2-phosphate-6-palmitate.

### TECHNICAL FIELD

The present invention relates to an external skin preparation and a production method of the same.

### BACKGROUND ART

Whitening agents have conventionally been incorporated in cosmetics and other external skin preparations. However, external skin preparations containing a whitening agent may cause irritation in persons with sensitive skin. This irritation normally refers to transitory irritation in which a substance that has contacted the skin surface passes through the skin barrier and reaches keratinocytes, thereby irritating the keratinocytes and inducing a local inflammatory reaction in the skin.

Consequently, various studies have been conducted in order to decrease the skin irritation of external skin preparations containing whitening agents.

For example, Patent Document 1 discloses an external skin preparation that combines a specific nonionic surfactant with a whitening agent in order to alleviate irritation.

Patent Document 2 discloses a cosmetic that combines an antibacterial phospholipid with a whitening agent in order to suppress the occurrence of transitory irritation.

Patent Document 3 discloses an external skin preparation for use as an external skin preparation that demonstrates superior whitening effects without having problems with stability, safety, smell and the like that combines a 2-O-alkyl ether derivative of L-ascorbic acid with one type or two or more types of whitening agents selected from the group consisting of L-ascorbic acid and salts thereof, ester derivatives of L-ascorbic acid and salts thereof, glycosides of L-ascorbic acid and salts thereof, alkoxysalicylic acids and salts thereof, glycosides of hydroquinone and salts thereof, tranexamic acid and derivatives thereof, derivatives of resorcinol, kojic acid, ellagic acid, linoleic acid and chamomile extract.

### CITATION LIST

### Patent Literature

PLT 1: Japanese Unexamined Patent Application, First Publication No. 2003-300857
PLT 2: Japanese Unexamined Patent Application, First Publication No. 2004-26657
PLT 3: Japanese Unexamined Patent Application, First Publication No. 2005-120023

### SUMMARY OF INVENTION

### Technical Problem

However, according to studies conducted by the inventors of the present invention, in the case of an external skin preparation containing a resorcinol derivative as a whitening agent, the occurrence of irritation cannot be adequately suppressed in the prior art. For example, skin irritation is not reduced even if a 2-O-alkyl ether derivative of L-ascorbic acid is incorporated as described in Patent Document 3.

With the foregoing in view, an object of the present invention is to provide an external skin preparation that contains a whitening agent in the form of a resorcinol derivative and causes minimal irritation of the skin, and a production method thereof.

### Solution to Problem

As a result of conducting extensive studies to solve the aforementioned problems, the inventors of the present invention found that skin irritation induced by a resorcinol derivative is alleviated by combining a whitening agent in the form of a resorcinol derivative with an ascorbyl 2-phosphate-6-palmitate, thereby leading to completion of the present invention.

The present invention is directed to:

[1] An external skin preparation comprising: 0.1% by weight to 5% by weight of a resorcinol derivative and 0.01% by weight to 5% by weight of an ascorbyl 2-phosphate-6-palmitate;
[2] The external skin preparation described in [1] above, wherein the weight ratio of the ascorbyl 2-phosphate-6-palmitate to the resorcinol derivative is 0.1 to 1;
[3] The external skin preparation described in [1] or [2] above, wherein the resorcinol derivative is 4-n-butylresorcinol;
[4] The external skin preparation described in any of [1] to [3] above, wherein the ascorbyl 2-phosphate-6-palmitate is sodium ascorbyl 2-phosphate-6-palmitate;
[5] The external skin preparation described in any of [1] to [4] above, which is a cosmetic;
[6] A production method of an external skin preparation, comprising: incorporating a resorcinol derivative and an ascorbyl 2-phosphate-6-palmitate such that the content of the ascorbyl 2-phosphate-6-palmitate in the external skin preparation is 0.01% by weight to 5% by weight, and the content of the resorcinol derivative is 0.1% by weight to 5% by weight; and
[7] The production method of an external skin preparation described in [6] above, wherein a weight ratio of the ascorbyl 2-phosphate-6-palmitate to the resorcinol derivative is 0.005 to 1.

Furthermore, ascorbyl 2-phosphate-6-palmitate is an ascorbic acid derivative that is known to be a compound that demonstrates effects such as whitening action, antioxidative action and collagen synthesis-promoting action. However, the ability of ascorbyl 2-phosphate-6-palmitate to suppress the occurrence of transitory irritation attributable to external skin preparations containing resorcinol derivatives as a result of incorporating therein is not disclosed or suggested in any of Patent Documents 1 to 3.

### Advantageous Effects of Invention

According to the present invention, an external skin preparation that contains a whitening agent in the form of a resorcinol derivative and only causes minimal skin irritation, and a production method of the same, can be provided.

### DESCRIPTION OF EMBODIMENTS

The external skin preparation of the present invention contains a resorcinol derivative and an ascorbyl 2-phosphate-6-palmitate.

<Resorcinol Derivative>

A resorcinol derivative known to be a whitening agent can be used for the resorcinol derivative, examples of which include alkylresorcinols, phenylethylresorcinol, chlororesorcinol and dimethoxytolyl propylresorcinol. From the viewpoint of whitening effects, the resorcinol derivative is preferably a 4-alkylresorcinol, more preferably a 4-alkylresorcinol in which the number of carbons of the alkyl group is 1 to 6, and particularly preferably 4-n-butylresorcinol.

One type of resorcinol derivative may be used alone or two or more types may be used in combination.

In the external skin preparation of the present invention, the content of the resorcinol derivative is 0.1% by weight to 5% by weight and preferably 0.3% by weight to 3% by weight based on the total weight of the external skin preparation. If the content of the resorcinol derivative is less than 0.1% by weight, whitening effects of the resorcinol derivative are not adequately obtained, while if the content of the resorcinol derivative exceeds 5% by weight, there are many cases in which whitening effects corresponding to the incorporated amount are not demonstrated.

<Ascorbyl 2-Phosphate-6-Palmitate>

The ascorbyl 2-phosphate-6-palmitate is a compound in which phosphoric acid is ester-bonded to a hydroxyl group bonded to a carbon atom at position 2 of ascorbic acid, palmitic acid is ester-bonded to a hydroxyl group bonded to a carbon atom at position 6, and which forms a salt by dissociation of a hydrogen atom from at least one of either the two hydroxyl groups bonded to the phosphorous atom in the phosphate group or the hydroxyl group bonded to a carbon atom at position 3 of ascorbic acid, and examples of salts thereof include sodium salts and potassium salts. For example, sodium ascorbyl 2-phosphate-6-palmitate is represented by the chemical formula indicated below.

The ascorbyl 2-phosphate-6-palmitate is preferably sodium ascorbyl 2-phosphate-6-palmitate since it offers ease of handling particularly in the case the external skin preparation is a cosmetic.

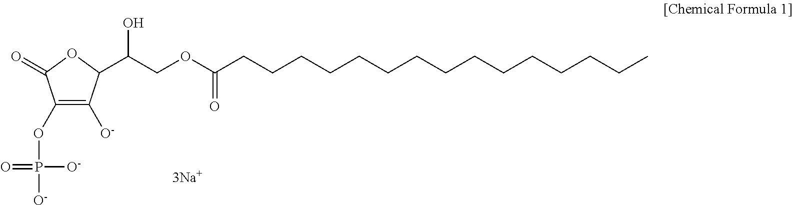 [SMILES: CCCCCCCCCCCCCCCC(=O)OCC(O)C1OC(=O)C(OP(=O)([O-])[O-])=C1[O-]]

One type of the ascorbyl 2-phosphate-6-palmitate may be used alone or two or more types may be used in combination.

In the external skin preparation of the present invention, the content of the ascorbyl 2-phosphate-6-palmitate is 0.01% by weight to 5% by weight and preferably 0.5% by weight to 3% by weight based on the total weight of the external skin preparation. Incorporation of this amount of the ascorbyl 2-phosphate-6-palmitate in the external skin preparation makes it possible to obtain an external skin preparation that demonstrates only minimal skin irritation. On the other hand, if the incorporated amount is less than 0.01% by weight, there is the risk of skin irritation caused by the resorcinol derivative being unable to be adequately suppressed. In addition, if the incorporated amount exceeds 5% by weight, suppressive effects do not increase corresponding to the incorporated amount, thereby making this uneconomical.

Moreover, within the ranges of the contents previously described, if the weight ratio of the ascorbyl 2-phosphate-6-palmitate to the resorcinol derivative is preferably made to be within the range of 0.005 to 1 and more preferably within the range of 0.1 to 0.5, an external skin preparation can be obtained that demonstrates even less skin irritation despite having an added a smaller amount of the ascorbyl 2-phosphate-6-palmitate.

<Other Arbitrary Components>

The external skin preparation of the present invention may also incorporate at least one type of component selected from ascorbic acid and salts thereof as well as ascorbic acid derivatives other than ascorbyl 2-phosphate-6-palmitate within a range that does not impair the effects of the present invention.

Examples of salts of ascorbic acid include sodium salts and potassium salts.

Examples of ascorbic acid derivatives other than ascorbyl 2-phosphate-6-palmitate include ascorbyl 3-phosphate-6-higher fatty acids and salts thereof, ascorbyl 6-higher fatty acids and salts thereof, ascorbyl 2,6-di-higher fatty acids and salts thereof, ascorbyl 2,3,5,6-tetra-higher fatty acids and salts thereof, ascorbyl 2-sulfuric acid and salts thereof and ascorbyl 2-glucosides. Examples of salts include sodium salts and potassium salts. Examples of higher fatty acids include higher fatty acids having 8 to 22 carbon atoms.

Specific examples of these ascorbic acid derivatives include sodium ascorbyl 3-phosphate-6-palmitate, ascorbyl 6-palmitate, ascorbyl 2,6-dipalmitate, ascorbyl 2,3,5,6-tetrapalmitate, disodium ascorbyl 2-sulfate and ascorbyl 2-glucoside.

The external skin preparation of the present invention may also contain as necessary components ordinarily used in external preparations within a range that does not impair the effects of the present invention, examples of which include carriers and additives pharmaceutically acceptable for use in external skin preparations.

Examples of such components include hydrocarbons, natural fats and oils, fatty acids, higher alcohols, alkyl glyceryl ethers, esters, silicone oils, polyvalent alcohols, monovalent lower alcohols, sugars, polymers, anionic surfactants, cationic surfactants, amphoteric surfactants, nonionic surfactants, natural surfactants, ultraviolet absorbers, powders, colorants, amino acids, peptides, vitamins, vitamin-like active substances, antiseptics, antioxidants, metal ion sequestering agents, moisturizers, antiphlogistics, pH adjusters, salts, organic acids, refined oils, terpenes, fragrances and water.

Examples of the external skin preparation of the present invention include cosmetics and pharmaceuticals.

In the case the external skin preparation of the present invention is a cosmetic, existing cosmetic raw materials can also be added at ordinary concentrations. For example, all cosmetic raw materials can be used that are described in the Second Edition of the Japanese Standards of Cosmetic Ingredients 1984 (Yakuji Nippo Ltd.), Japanese Cosmetic Ingredients Codex, Ministry of Health, Labour and Welfare, Pharmaceutical Affairs Bureau, Inspection Section, ed., 1993 (Yakuji Nippo Ltd.), Supplement to the Japanese Cosmetic Ingredients Codex, Ministry of Health, Labour and Welfare, Pharmaceutical Affairs Bureau, Inspection Section, ed., 1993 (Yakuji Nippo Ltd.), Comprehensive Licensing Standards of Cosmetics by Category, Ministry of Health, Labour and Welfare, Pharmaceutical Affairs Bureau, Inspection Section, ed., 1993 (Yakuji Nippo Ltd.), Categorized Japanese Cosmetic Ingredients Codex, Ministry of Health, Labour and Welfare, Pharmaceutical Affairs Bureau, Inspection Section, ed., 1997 (Yakuji Nippo Ltd.) and the Encyclopedia of Chemical Raw Materials, 1991 (Nikko Chemicals Co., Ltd.).

There are no particular limitations on the form of the external skin preparation of the present invention provided it is used by contacting the skin at the time of use, and is suitably determined corresponding to the application. Examples of forms that can be applied include a lotion, milky liquid, cream or facial pack.

The external skin preparation of the present invention is produced by incorporating and formulating the resorcinol derivative, the ascorbyl 2-phosphate-6-palmitate and other arbitrary components. Formulation can be carried out in accordance with ordinary methods corresponding to the form of the preparation.

Incorporating the resorcinol derivative and the ascorbyl 2-phosphate-6-palmitate in the manner described above allows the obtaining of an external skin preparation that demonstrates minimal skin irritation by suppressing the occurrence of transitory skin irritation caused by the resorcinol derivative.

Consequently, the present invention is useful over the entire range of external skin preparations, including cosmetics and pharmaceuticals, and is particularly useful in cosmetics.

Although the following provides a more detailed explanation of the present invention based on examples thereof, the present invention is not limited to these examples.

Skin irritation of the external skin preparations (lotions) obtained in each of the subsequently described examples, comparative examples and reference examples was evaluated according to the procedure described below.

<Skin Irritation Evaluation Method>

Skin irritation was investigated for the resulting lotions by the 24-hour closed patch method using a guinea pig skin damage model (Hartley white, females, 300 g to 400 g, prepared by stripping the skin three times with tape after shaving) in groups of 5 animals each. Draize criteria were used to evaluate skin irritation. Namely, skin irritation was evaluated based on the criteria indicated below.
Score=2: Reaction with edema
Score=1: Reaction with obvious erythema
Score=0.5: Reaction with possible erythema
Score=0: No reaction

### Example 1, Comparative Examples 1 to 3, and Reference Example 1

Lotions having the compositions indicated in Table 1 (units: wt %) were prepared according to the procedure described below.

4-n-butylresorcinol was warmed and dissolved in purified water and ethanol while other components were dissolved at room temperature followed by mixing to prepare lotions.

Skin irritation was evaluated using the resulting lotions. The results are shown in Table 2. The numerical values indicated in Table 2 indicate the number of guinea pig damaged skin models that corresponded to the respective scores.

TABLE 1

| | | Comp. | Comp. | Comp. | Ref. |
|---|---|---|---|---|---|
| Components | Ex. 1 | Ex. 1 | Ex. 2 | Ex. 3 | Ex. 1 |
| Ethyl alcohol (95%) | 25 | 25 | 25 | 25 | 25 |
| POE(25) hydrogenated | 2 | 2 | 2 | 2 | 2 |
| castor oil ether | | | | | |
| Methyl | 0.1 | 0.1 | 0.1 | 0.1 | 0.1 |
| p-hydroxybenzoate | | | | | |
| 4-n-butylresorcinol | 0.5 | 0.5 | 0.5 | 0.5 | |
| 2-O-methyl ascorbic acid | | 0.5 | | | |
| Sodium ascorbyl | | | 0.5 | | |
| 2-phosphate | | | | | |
| Sodium ascorbyl | 0.5 | | | | |
| 2-phosphate-6-palmitate | | | | | |
| Glycerin | 2 | 2 | 2 | 2 | 2 |
| Propylene glycol | 1 | 1 | 1 | 1 | 1 |
| Purified water | 68.9 | 68.9 | 68.9 | 69.4 | 69.9 |
| Total | 100 | 100 | 100 | 100 | 100 |

TABLE 2

| | | Comp. | | | |
|---|---|---|---|---|---|
| | Ex. 1 | Ex. 1 | Comp. Ex. 2 | Comp. Ex. 3 | Ref. Ex. 1 |
| Score = 2 | 0 | 0 | 0 | 0 | 0 |
| Score = 1 | 0 | 3 | 2 | 3 | 0 |
| Score = 0.5 | 0 | 2 | 3 | 2 | 0 |
| Score = 0 | 5 | 0 | 0 | 0 | 5 |

As indicated by the above results, although Comparative Example 3, in which 4-n-butylresorcinol was incorporated in the composition of Reference Example 1 that did not demonstrate skin irritation, demonstrated skin irritation, Example 1, which further incorporated sodium ascorbyl 2-phosphate-6-palmitate, did not demonstrate skin irritation in the same manner as Reference Example 1.

On the other hand, Comparative Example 1, which incorporated 2-O-methyl ascorbic acid instead of sodium ascorbyl 2-phosphate-6-palmitate, demonstrated skin irritation in the same manner as Comparative Example 3. In addition, Comparative Example 2, which incorporated sodium ascorbyl 2-phosphate instead of sodium ascorbyl 2-phosphate-6-palmitate, showed an unacceptable level of skin irritation although skin irritation improved in comparison with Comparative Example 3.

### Examples 2 to 4, Comparative Example 4, and Reference Example 2

Lotions having the compositions indicated in Table 3 (units: wt %) were prepared according to the procedure described below.

First, the following compositions A, B and C were prepared.

Composition A: 4-n-butyl resorcinol and/or sodium ascorbyl 2-phosphate-6-palmitate were heated and dissolved followed by mixing into a mixture of other components (oily components) and holding at 80° C.

Composition B: Each component was dissolved in heated purified water and held at 80° C. (B phase).

Composition C: Arginine was dissolved in purified water.

Next, Composition A (oily phase) was added to Composition B (aqueous phase) followed by preliminarily emulsifying and then uniformly emulsifying with a homomixer. Following emulsification, the emulsion was cooled to 30° C. followed by the addition of Composition C to obtain lotions.

Skin irritation was evaluated using the resulting lotions. The results are shown in Table 4.

TABLE 3
Lotion Composition (units: wt %)

| | | Ex. | Ex. | Ex. | Comp. | Ref. |
|---|---|---|---|---|---|---|
| | Components | 2 | 3 | 4 | Ex. 4 | Ex. 2 |
| Compo- | Behenyl alcohol | 0.3 | 0.3 | 0.3 | 0.3 | 0.3 |
| sition | Glycerin stearate | 2.0 | 2.0 | 2.0 | 2.0 | 2.0 |
| A | PEG-60 glyceryl | 0.5 | 0.5 | 0.5 | 0.5 | 0.5 |
| | isostearate | | | | | |
| | Squalane | 5.0 | 5.0 | 5.0 | 5.0 | 5.0 |
| | Octamethylcyclotetra- | 2.0 | 2.0 | 2.0 | 2.0 | 2.0 |
| | siloxane | | | | | |
| | Triethylhexanoin | 1.0 | 1.0 | 1.0 | 1.0 | 1.0 |
| | Ethylhexyl palmitate | 1.5 | 1.5 | 1.5 | 1.5 | 1.5 |
| | Propyl p-hydroxy- | 0.1 | 0.1 | 0.1 | 0.1 | 0.1 |
| | benzoate | | | | | |
| | 4-n-butylresorcinol | 2.0 | 2.0 | 2.0 | 2.0 | |
| | Sodium ascorbyl 2- | 1.0 | 0.2 | 0.01 | | |
| | phosphate-6-palmitate | | | | | |
| Compo- | 1,3-butyleneglycol | 4.0 | 4.0 | 4.0 | 4.0 | 4.0 |
| sition | Glycerin | 4.0 | 4.0 | 4.0 | 4.0 | 4.0 |
| B | Carboxyvinyl polymer | 0.2 | 0.2 | 0.2 | 0.2 | 0.2 |
| | Xanthane gum | 0.2 | 0.2 | 0.2 | 0.2 | 0.2 |
| | Methyl p-hydroxy- | 0.2 | 0.2 | 0.2 | 0.2 | 0.2 |
| | benzoate | | | | | |
| | Purified water | 70.9 | 71.7 | 71.9 | 71.9 | 73.9 |
| Compo- | Arginine | 0.1 | 0.1 | 0.1 | 0.1 | 0.1 |
| sition | Purified water | 5.0 | 5.0 | 5.0 | 5.0 | 5.0 |
| C | | | | | | |

| | | | | | |
|---|---|---|---|---|---|
| Total | 100.0 | 100.0 | 100.0 | 100.0 | 100.0 |

TABLE 4

| | Ex. 2 | Ex. 3 | Ex. 4 | Comp. Ex. 4 | Ref. Ex. 2 |
|---|---|---|---|---|---|
| Score = 2 | 0 | 0 | 0 | 0 | 0 |
| Score = 1 | 0 | 0 | 0 | 3 | 0 |
| Score = 0.5 | 0 | 0 | 2 | 2 | 0 |
| Score = 0 | 5 | 5 | 3 | 0 | 5 |

As indicated by the above results, Comparative Example 4, which incorporated 4-n-butyl resorcinol in the composition of Reference Example 2 that did not demonstrate skin irritation, demonstrated skin irritation. The skin irritation was alleviated by Example 4 that incorporated 0.01% by weight of sodium ascorbyl 2-phosphate-6-palmitate. In contrast to these, Examples 2 and 3, which incorporated increased amounts of sodium ascorbyl 2-phosphate-6-palmitate, did not demonstrate skin irritation in the same manner as Reference Example 2.

### Example 5, Comparative Examples 5 and 6, and Reference Example 3

Lotions having the compositions indicated in Table 5 (units: wt %) were prepared according to the procedure described below.

First, the following compositions A and B were prepared.

Composition A: Each component was heated and dissolved, mixed and held at 70° C.

Composition B: Purified water and ethanol were mixed, heated and dissolved in 4-n-butylresorcinol followed by dissolving the other components therein and holding at 70° C.

Next, Composition A (oily phase) was added to Composition B (aqueous phase) followed by preliminarily emulsifying and then uniformly emulsifying with a homomixer. Following emulsification, the emulsion was cooled to 30° C. while stirring well to obtain lotions.

Skin irritation was evaluated using the resulting lotions. The results are shown in Table 6.

TABLE 5

| | | | Comp. | Comp. | Ref. |
|---|---|---|---|---|---|
| | Components | Ex. 5 | Ex. 5 | Ex. 6 | Ex. 3 |
| Com- | POE(20) cetyl ether | 1.0 | 1.0 | 1.0 | 1.0 |
| position A | Silicone oil | 2.0 | 2.0 | 2.0 | 2.0 |
| | Liquid paraffin | 3.0 | 3.0 | 3.0 | 3.0 |
| Com- | Purified water | 68.9 | 69.4 | 68.9 | 71.4 |
| position B | Ethanol | 15.0 | 15.0 | 15.0 | 15.0 |
| | 4-n-butylresorcinol | 2.0 | 2.0 | 2.0 | |
| | Propylene glycol | 5.0 | 5.0 | 5.0 | 5.0 |
| | Glycerin | 2.0 | 2.0 | 2.0 | 2.0 |
| | Carboxyvinyl polymer | 0.3 | 0.3 | 0.3 | 0.3 |
| | Hydroxypropyl cellulose | 0.1 | 0.1 | 0.1 | 0.1 |
| | 2-aminomethyl propanol | 0.1 | 0.1 | 0.1 | 0.1 |
| | Methyl p-hydroxybenzoate | 0.1 | 0.1 | 0.1 | 0.1 |
| | 2-O-methyl ascorbic acid | | | 0.5 | |
| | Sodium ascorbyl | 0.5 | | | |
| | 2-phosphate-6-palmitate | | | | |

| | | | | |
|---|---|---|---|---|
| Total | 100.0 | 100.0 | 100.0 | 100.0 |

TABLE 6

| | Ex. 5 | Comp. Ex. 5 | Comp. Ex. 6 | Ref. Ex. 3 |
|---|---|---|---|---|
| Score = 2 | 0 | 0 | 0 | 0 |
| Score = 1 | 0 | 2 | 3 | 0 |
| Score = 0.5 | 0 | 3 | 2 | 0 |
| Score = 0 | 5 | 0 | 0 | 5 |

As indicated by the above results, Comparative Example 5, which incorporated 4-n-butyl resorcinol in the composition of Reference Example 3 that did not demonstrate skin irritation, demonstrated skin irritation. Example 5, which incorporated sodium ascorbyl 2-phosphate-6-palmitate, did not demonstrate skin irritation in the same manner as Reference Example 3.

On the other hand, Comparative Example 6, which incorporated 2-O-methyl ascorbic acid instead of sodium ascorbyl 2-phosphate-6-palmitate, demonstrated even more severe skin irritation than Comparative Example 5.

Furthermore, among the raw materials used in each of the aforementioned examples, comparative examples and reference examples, “POE” in the POE(25) hydrogenated castor oil ether and POE(20) cetyl ether is an abbreviation of polyoxyethylene. The numerical value shown in parentheses after POE indicates the average degree of polymerization of oxyethylene groups in the POE units.

In the PEG-60 of PEG-60 glycerin isostearate, PEG indicates a polyoxyethylene chain, while the numerical value indicated thereafter indicates the average degree of polymerization of the oxyethylene groups.

CARBOP1 980 (trade name, Noveon Corp.) was used for the carboxyvinyl polymer.

Keltrol CG-SFT (Sansho Co., Ltd.) was used for the xanthane gum.

Silicone KF96 (20 cs) (trade name, Shin-Etsu Chemical Co., Ltd.) was used for the silicone oil.

1.-7. (canceled)

8. An external skin preparation comprising:
0.1% by weight to 5% by weight of a resorcinol derivative, and
0.01% by weight to 5% by weight of an ascorbyl 2-phosphate-6-palmitate.

9. The external skin preparation according to claim 8, wherein the weight ratio of the ascorbyl 2-phosphate-6-palmitate to the resorcinol derivative is 0.005 to 1.

10. The external skin preparation according to claim 8, wherein the resorcinol derivative is 4-n-butylresorcinol.

11. The external skin preparation according to claim 8, wherein the ascorbyl 2-phosphate-6-palmitate is sodium ascorbyl 2-phosphate-6-palmitate.

12. A production method of an external skin preparation, comprising:
incorporating a resorcinol derivative and an ascorbyl 2-phosphate-6-palmitate into the preparation such that the content of the resorcinol derivative in the external skin preparation is 0.1% by weight to 5% by weight, and the content of the ascorbyl 2-phosphate-6-palmitate is 0.01% by weight to 5% by weight.

13. The production method of an external skin preparation according to claim 12, wherein a weight ratio of the ascorbyl 2-phosphate-6-palmitate to the resorcinol derivative is 0.005 to 1.
